(19) United States
(12) Patent Application Publication
(10) Pub. No.: US 2008/0220526 A1
(43) Pub. Date: Sep. 11, 2008

(54) Gum coatings for cell culture, methods of manufacture and methods of use

(76) Inventors: Adam J. Ellison, Painted Post, NY (US); Wageesha Senaratne, Horseheads, NY (US); Ying Wei, Painted Post, NY (US)

Correspondence Address: CORNING INCORPORATED, SP-TI-3-1, CORNING, NY, 14831, US

(21) Appl. No.: 12/075,079

(22) Filed: Mar. 7, 2008

Related U.S. Application Data
(60) Provisional application No. 60/906,168, filed on Mar. 9, 2007.

(51) Int. Cl. C12N 5/06; C12M 1/00; B05D 3/00

(52) U.S. Cl. 435/402; 435/305.1; 427/398.1

(57) ABSTRACT

This invention relates to coatings for cell culture surfaces. More particularly, this invention relates to coatings for cell culture surfaces which are derived from or contain gums including naturally occurring gums, plant gums, galactomannan gums or derivatives thereof. The invention also relates to articles of manufacture (e.g., cell culture vessels and labware) having such coatings, methods of applying these coatings to cell culture surfaces, and methods of using coated cell culture vessel.

### CROSS-REFERENCE TO RELATED APPLICATIONS

This Application claims the benefit of U.S. Provisional Application Ser. No. 60/906,168 filed Mar. 9, 2007 and entitled “Coatings for Cell Culture Surfaces” which is incorporated by reference herein. This Application is related to Application Serial No. ______ filed on ______ entitled “Three Dimensional Gum Matrices for Cell Culture, Manufacturing Methods and Methods of Use.”

### FIELD

This invention relates to coatings for cell culture surfaces. More particularly, this invention relates to coatings for cell culture surfaces which are derived from or contain gums including naturally occurring gums, plant gums, galactomannan gums or derivatives thereof. The invention also relates to articles of manufacture (e.g., cell culture vessels and labware) having such coatings, methods of applying these coatings to cell culture surfaces, and methods of using coated cell culture vessels.

### BACKGROUND

In vitro culturing of cells provides material necessary for cell biology research, and provides much of the basis for advances in the fields of pharmacology, physiology and toxicology. However, isolated cultured eukaryotic cells living in an incubator in a culture vessel bathed in cell culture media often have very different characteristics compared to individual cells in vivo. Information obtained from experiments conducted on primary and secondary cultures of eukaryotic cells is informative to pharmacologists, physiologists and toxicologists only to the extent that cultured cells have the same characteristics as intact cells.

Cells can grow on surfaces of cell culture vessels. For example, cells in liquid media can be introduced into a cell culture vessel such as a cell culture flask or a multi-well cell culture plate, the cell culture vessel placed into a suitable environment such as an incubator, and the cells allowed to settle onto a surface of the cell culture vessel where they attach, grow, and divide. However, some cells perform better than others when growing on a flat surface. Some cells require different surfaces in order to maintain a more natural phenotype, and to provide optimal in vitro data.

Conditions of cell culture affect the characteristics of cells in culture, and therefore affect the value of data obtained from cells in culture. There is a need in the industry to provide cell culture surfaces and conditions to provide data that is more highly correlated with in vivo cell behavior.

### SUMMARY

The present invention relates to a cell culture surface coating made from at least one gum material, including naturally occurring gums, plant gums, galactomannan gums or derivatives thereof. In an embodiment, the invention relates to a cell culture surface coating made from at least one galactomannan gum. Galactomannan gums include locust bean gum, (also known as carob bean gum, carob seed gum, carob gum), guar gum, cassia gum, tara gum, mesquite gum, fenugreek gum, and their derivatives. In embodiments, the gum material can be processed, purified, chemically modified, treated, or subjected to heating or freeze-thaw cycles. In embodiments, the gum coating can be in the form of a gel coating, a hydrogel coating, a film coating or a hydrofilm coating. In additional embodiments, the cell culture surface coating is tunable. In additional embodiments, the gum coating can further include a biologically active compound.

In yet another embodiment, the cell culture surface coating of the present invention can be made of more than one gum material. The combined gum material can include locust bean gum (also known as carob bean gum, carob seed gum, carob gum), guar gum, cassia gum, tragacanth gum, tara gum, karaya gum, gum acacia (gum Arabic), ghatti gum, cherry gum, apricot gum, tamarind gum, mesquite gum, larch gum, psyllium, fenugreek gum, xanthan gum, seaweed gum, gellan gum, agar gum, cashew gum, carrageenan and curdlan.

In an embodiment, the present invention also includes a cell culture vessel for eukaryotic cell culture comprising a substrate and a coating on the substrate wherein the coating comprises at least one galactomannose gum. In an embodiment of the present invention, the cell culture vessel can be plastic or glass. In a further embodiment of the present invention, the cell culture vessel can be labware.

In yet another embodiment, the present invention also includes a method of making a cell culture vessel which includes the steps of preparing a galactomannan gum solution, and applying the galactomannan gum solution to the cell culture vessel surface. In additional embodiments, the method of making the cell culture vessel of the present invention also includes steps of heating the cell culture vessel or subjecting the cell culture vessel in freeze/thaw cycles. In additional embodiments, the present invention includes methods of using the coated cell culture vessels of the present invention to culture cells.

### BRIEF DESCRIPTION OF THE DRAWINGS

The invention is best understood from the following detailed description when read with the accompanying figures.

FIG. 1 is an illustration of an embodiment of the cell culture coating of the present invention.

FIGS. 2A, B and C are modified photomicrographs showing bright field (FIG. 2A), live staining (FIG. 2B) and dead staining (FIG. 2C) of HepG2/C3A cells 9 days after being cultured on embodiments of the locust bean gum-coated surfaces of the present invention.

FIGS. 3A, B and C are modified photomicrographs illustrating actin filament staining of HepG2/C3A cells with Texas Red-phalloidin of cells 9 days after being cultured on distinct surfaces.

FIGS. 4A, B and C are modified photomicrographs illustrating images of HepG2/C3A cells grown on embodiments of cell culture coated surfaces of the present invention in bright field (FIG. 3A), and after Live/Dead staining (FIGS. 3B and 3C).

FIGS. 5A, B and C illustrate a bright field photomicrograph shown at increasing magnifications illustrating HepG2/C3A cells grown on embodiment of the cell culture coating of the present invention.

FIG. 6 is a chart illustrating cell growth following hepatocyte culture on coated cell culture surfaces of the present invention compared to uncoated TCT culture plates.

FIG. 7 is a chart illustrating the results of albumin assays performed on HepG2/C3A cells grown on locust bean gum coating embodiments of the present invention compared to Matrigel™ collagen I, and TCT.

FIG. 8 is a chart illustrating albumin production evaluations of HepG2/C3A cells cultured on distinct substrates with Matrigel™, Collagen coated dishes and TCT as controls.

FIG. 9 illustrates albumin production evaluations at three different times (7, 10, and 14 days culturing) for TCT, locust bean gum-coating embodiments of the present invention, a blend of guar gum and carrageenan-coating embodiment of the present invention, and a blend of guar gum and curdlan-coating cell culture surface embodiments of the present invention.

### DETAILED DESCRIPTION

Embodiments of the present invention provide cell culture coatings which provide a cell culture environment that is favorable for cell growth and cell physiology. In embodiments, the present invention provides cell culture surface coatings made from naturally occurring branched galactose based polysaccharides. These naturally occurring branched galactose based polysaccharides include locust bean gum (also known as carob bean gum, carob seed gum, carob gum), guar gum, cassia gum, tragacanth gum, tara gum, karaya gum, gum acacia (gum Arabic), ghatti gum, cherry gum, apricot gum, tamarind gum, mesquite gum, larch gum, psyllium, fenugreek gum, xanthan gum, seaweed gum, gellan gum, agar gum, cashew gum, carrageenan and curdlan. In additional aspects, the present invention includes methods of manufacturing the cell culture surface coatings of the present invention, and methods of using these cell culture surface coatings in cell culture vessels and containers to provide preferable cell culture conditions.

In the following detailed description, for purposes of explanation and not limitation, exemplary embodiments disclosing specific details are set forth in order to provide a thorough understanding of the present invention. However, it will be apparent to one having ordinary skill in the art that the present invention may be practiced in other embodiments that depart from the specific details disclosed herein. In other instances, detailed descriptions of well-known devices and methods may be omitted so as not to obscure the description of the present invention. This cell culture environment may be appropriate for any type of cell in culture including primary cells, immortalized cells lines, groups of cells, tissues in culture, adherent cells, suspended cells, cells growing in groups such as embryoid bodies, eukaryotic cells, prokaryotic cells, or any other cell type.

Many cells have special requirements in culture. These cell culture requirements or preferences are exhibited by the cells as they take on different cell morphologies in culture, change their regenerative characteristics, and change their metabolic and secretory characteristics. Embryonic stem cells, for example, require cell culture conditions which allow the cells to grow and propagate in an undifferentiated state until they are exposed to chemical and/or physical signals that allow them to differentiate into a desired cell type. Heart cells, for example, grown in an appropriate in vitro environment, will beat in unison with neighboring cells. For cells growing in culture to have characteristics in vitro that are relevant to their characteristics in vivo, cell culture conditions must be carefully controlled.

Hepatocytes, for example, seem to have very specific cell culture requirements in order to maintain important in vivo characteristics in vitro. Hepatocytes are the primary functional cells of the liver and perform an amazing number of metabolic, endocrine and secretory functions. Hepatocytes make up 60-80% of the cytoplasmic mass of the liver. They are exceptionally active in synthesis of proteins, cholesterol, bile salts and phospholipids for export, and are involved in protein storage and transformation of carbohydrates. In vivo, hepatocytes are responsible for detoxification, or modification and excretion of exogenous and endogenous substances from the body.

Primary and secondary cultures of hepatocytes have been used for pharmacology, toxicology and physiology studies, for studying the mechanisms of liver regeneration and differentiation, as well as for understanding factors which affect characteristics of hepatocytes in culture. Historically, primary hepatocytes have exhibited a limited replicating lifespan in culture. In addition, when stimulated to divide in culture they have generally lost differentiated functions such as the ability to synthesize and secrete albumin and transferrin. When cultured appropriately, cultured hepatocyte cells self-assemble into spheroidal structures that enhance liver-specific functions. When hepatocytes are not cultured appropriately, they form flat cells or cell clumps. The level of liver-specific activities, including albumin secretion and P450 activity, are lower with flat cell or cell clumps than those with spheroids.

When culturing cells of any cell type, a preferable cell culture environment promotes desirable cell characteristics in vitro. Such an environment that can be provided in a reproducible and inexpensive manner is desirable. In addition, cell culture vessels which incorporate these desirable environments are needed. And, methods of manufacturing and using cell culture vessels that incorporate desirable cell culture environments are needed.

Embodiments of the present invention include coatings for cell culture surfaces, containers or vessels. In one embodiment of the present invention, the coating is derived from at least one gum. While gums are discussed herein, it will be understood by those in the art that the term “gum” includes any composition containing plant gums or plant seed gums obtainable from natural plant gum sources, from recombinant sources, from synthetic sources, or from combinations of natural sources, recombinant sources and synthetic sources. Further, “gum” includes mixtures of various gums from different sources as well as gum compositions containing additional ingredients. Further, these gums can be modified by enzymatic or chemical manipulations to enhance desired chemical characteristics.

Naturally occurring gums are found in natural products. Examples of naturally occurring gums derived from plants include guar gum, locust bean gum (also known as carob bean gum, carob seed gum and carob gum), cassia gum, tragacanth gum, tara gum, karaya gum, gum acacia (also known as gum arabic), ghatti gum, cherry gum, apricot gum, tamarind gum, mesquite gum, larch gum, psyllium, fenugreek gum and tara gum. Gums that are derived from bacterial and algal sources include xanthan gum, seaweed gum, gellan gum, agar gum, cashew gum, carrageenan and curdlan. It will be understood by those of skill in the art that naturally occurring gums include mixtures of naturally occurring gums with various gums from different sources, and gums which have been collected from natural sources and then chemically purified, treated, modified, tuned and/or mixed with other ingredients to form suitable coatings in embodiments of the present invention.

Naturally occurring gums can be produced as exudates of plants which may be produced by plants after a plant has been wounded. A wounded gum-producing plant produces an exudate in response to a wound (tears of gum).

These kinds of plant exudates may be harvested by wounding a plant or a plant seed and removing the exudates. These exudates can then be cleaned to remove dust, dirt and other impurities. The exudates may be dried, powdered and suspended in liquid. Further processing steps may include enzymatic treatment, filtration, centrifugation, hydrolysis and other chemical modifications.

Plant gums can also be derived from plant seeds. Seed-derived gums include guar (Cyamopsis tetragonoloba), locust bean or carob bean (Ceratonia siliqua), tara (Caesalpinia spinosa), fenugreek (Trigonella foenum-graecom), mesquite gum (Prosopis spp) and Cassia gum (Cassia tora). These are generally sub-tropical plants.

Gums are generally derived from the endosperm of the seeds. After removal of the shell and the germ of the seed, the endosperm is ground. The resulting powder (flour) can contain a high polysaccharide content which, upon mixing with water and/or other ingredients, can form a liquid gum substance. The suspension formed upon mixing the powdered gum with liquid can contain impurities such as cellulose, other plant matter, and various chemical impurities. The suspension can be treated to increase the purity of the gum suspension. For example, the liquid can be centrifuged, filtered, heated, or put through freeze-thaw cycles to remove impurities. An embodiment of the present invention includes a gum which has been purified by one of these steps, or by other means known in the art. An embodiment of the present invention includes a gum which has been purified. Additional embodiments of the present invention include plant gums, naturally occurring gums and galactomannan gums which have been purified.

Powdered gum raw materials may also be purified or filtered according to particle size. For example sieves can be used to separate powdered gum material according to particle size. Particle sieve size openings of 250, 106, 53, 38 and 25 μm can be used to separate powdered gum material to create filtered particle size mixtures of between 106 and 250 μm, between 53 and 106 μm, between 38 and 53 μm, and between 25 and 38 μm particles. These separated particle sizes, or mixtures of these particle sizes, can be used to produce embodiments of the cell culture coatings of the present invention.

Plant gums have been used for centuries. Ancient Egyptians used locust bean gum to bind the wrapping of mummies. Plant gums are used extensively commercially as food additives for their excellent properties including thickening, preventing the formation of ice crystals in frozen foods, retaining crispiness, retaining water, temperature tolerance and pH tolerance. Plant gums are used in the cosmetics industry as thickeners as hydrating agents, and as emulsification agents or foam stabilizers. Some gums, galactomannan gums for example, are non-ionic, and therefore are not affected by ionic strength or pH. They will degrade at pH and temperature extremes. In general, these conditions are not present in cell culture, and therefore do not pose a concern in cell culture applications.

Gum material can be purchased from chemical supply houses including Sigma-Aldrich, Fluka, TIC-Gums Inc. (Belcamp, Md.), Gum Technology Corporation and Herchles (formerly Aqualon) Wilmington, Del. Because these gums are used as food additives, and for industrial applications, these materials may be provided in less pure forms than might be necessary for laboratory uses. Therefore, additional purification steps and additional treatments may be necessary. Purification steps may include, for example, extraction in a solvent that is less polar than water, for example, ethanol. However, in the examples below, both purified and unpurified materials were utilized with favorable results.

Locust bean gum is a galactomannan polysaccharide of the following formula:

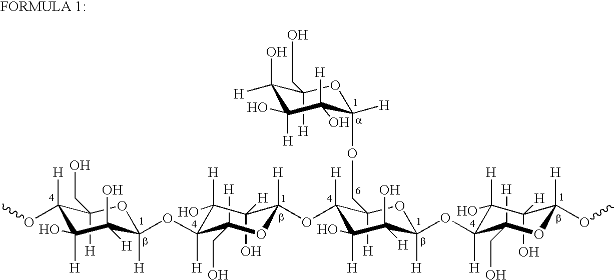

Locust bean gum (LBG) is a galactomannan polysaccharide consisting of mannopyranose backbone with branchpoints from their 6-positions linked to α-D-galactose residues. Locust bean gum has about 4 mannose residues for every galactose residue (a mannose/galactose ratio of about 4). Galactomannan gums include locust bean gum (LBG), guar gum, cassia gum, tara gum, mesquite gum, and fenugreek gum. Guar gum is also a galactomannan polysaccharide consisting of a mannopyranose backbone. However, guar gum has more galactose branchpoints than locust bean gum. Guar gum has a mannose/galactose ratio of about 2. The mannose/galactose ratio is about 1:1 for mesquite gum and fenugreek gum, about 3:1 for tara gum and about 5:1 for Cassia gum. This difference in structure causes guar gum to be more soluble have a higher viscosity than locust bean gum. That is, locust bean gum is a galactomannan compound with lower galactose substitution and therefore it is “less stiff”. The larger the mannose/galactose ratio, the less viscous and less soluble the gum. Therefore, gums with a higher mannose/galactose ratio are less stiff and, more flexible, while gums with a lower mannose/galactose ratio are better subject to solubility, dispersion and emulsification. Locust bean gum has more flexibility (lower modulus) than guar gum. Higher galactose substitution of these gums gives them improved solubility, dispersiveness and emulsification. Higher galactose substitution makes the galactomannan polysaccharides stiffer. Higher substitution lends the gum characteristics of higher viscosity and higher solubility.

Galactomannan gums are gums which may be derived from natural sources, or from recombinant or synthetic sources, which are galactomannan polysaccharides. Galactomannan gums also include these gums which are purified or treated or modified by processes which may include enzymatic treatment, filtration, centrifugation, hydrolysis, freeze-thaw cycles, heating and chemical treatments or modifications, mixtures of galactomannan gums with other galactomannan gums or non-galactomannan gums, and mixtures of galactomannan gums with other ingredients which optimize the cell culture characteristics of embodiments of coatings of the present invention.

Gums, including galactomannan gums, may be derived from recombinant or synthetic sources. For example, galactomannose may be synthesized in vivo from GDP-mannose and UDP-galactose by the enzymes mannan synthase and galactosyltransferase. DNA coding for these proteins has been isolated and characterized, (US Patent Publication 2004/0143871) and recombinant plants transformed with these enzymes have been shown to express elevated levels of galactomannan. In addition, the degree of galactosylation of the mannopyranose backbone may be influenced by the presence (or absence) of alpha-galactosidase in vivo, (see Edwards et al. Plant Physiology (2004) 134: 1153-1162). Alpha-galactosidase removes galactose residues from the mannopyranose backbone. For example, seeds that naturally express galactomannans with a lower degree of galactosylation may express (or express more) alpha galactosidase, which removes galactose moieties from the mannopyranose backbone in those species of plant. The alpha-galactosidase enzyme may be used to reduce the presence of galactose on the mannopyranose backbone of naturally occurring galactomannose gums in a laboratory manipulation of the characteristics of the naturally occurring galactomannose gum. Embodiments of the present invention include gums, including naturally occurring gums and galactomannose gums, which have been treated with alpha-galactosidase to reduce the presence of galactose on the mannopyranose backbone. Embodiments of the present invention include gums which have been treated with alpha-galactosidase or other enzymes or chemical treatments, to “tune” the gums to provide the gum with desired characteristics as a coating for cell culture surfaces.

Without being held to any particular theory, galactomannan gums, with their mannose backbones and galactose sidechains, have been identified by the inventors as providing a surface coating for cell culture which presents galactose moieties to cells in culture. For some cell types, these galactose moieties may provide a surface or growth bed which encourages the growth of cells in culture.

Cultured hepatocytes may respond favorably to the presence of galactose in culture. The density and orientation of galactose moieties may regulate the attachment of cells such as hepatocytes to a cell culture substrate and improve the function of the cells in vitro. This may occur through the inhibition of integrin clustering and/or controlling cell-substrate and cell-cell interactions. The binding of multivalent galactose as a specific ligand to the asialoglycoprotein receptors (ASGPRs) on the surface of hepatocytes has been extensively studied and has been shown to improve hepatocyte adhesion while maintaining viability. Although ASGPR does not physiologically function as an adhesion receptor, galactose-containing polymers have been used to induce the selective adhesion of primary hepatocytes to the substrate (Weigel, P H. Rat hepatocytes bind to synthetic galactoside surface via a patch of asialoglycoprotein receptors. J Cell Biol 1980;87:855-61; Kobayashi A, et al., Enhanced adhesion and survival efficacy of liver cells in culture dishes coated with a lactose-carrying styrene homopolymer. Macromol Chem Rapid Commun 1986;7:645-50 and Gutsche A T, et al. N-acetylglucosamine and adenosine derivatized surfaces for cell culture: 3T3 fibroblast and chicken hepatocyte response. Biotechnol. Bioeng 1994;43:801-9).

The hepatocyte cell-cell interactions may result in promotion of the formation of spheroidal aggregates of hepatocytes in culture which in turn promotes the formation of bile canaliculi, gap junctions, tight junctions, and E-cadherins, resulting in enhanced hepatocyte function, and improved hepatocyte culture performance.

Embodiments of the present invention include gum compounds which can be modified to optimize physical and chemical characteristics of cell culture coatings made from the gum compounds. Physical and chemical characteristics of the cell culture coatings of the present invention can be “tuned” by chemically modifying the gum material. “Tuned” or “tunable” used here means that gum material, obtained naturally or synthetically, can be modified, either physically or chemically to adjust the physical or chemical characteristics of the material to provide a cell culture substrate that promotes a desired cell culture environment. This cell culture substrate may be tuned to provide a preferred cell culture environment for a particular cell type growing in a particular type of cell culture media, for a particular purpose. Physical characteristics of the material can be changed by changing the porosity of the coating material, changing the modulus of the coating, changing the charge density and distribution, surface energy, topology and porosity, or by changing other physical characteristics of the matrix. Chemical characteristics of the material can be changed by providing chemical crosslinkers in the material, adding or removing chemical groups from a particular matrix material, mixing different gum substances together, changing the type and density of receptor ligands present in the cell culture environment or by other chemical modifications. Chemical modifications can change the physical characteristics of embodiments of the present invention, and physical modifications can involve chemical modifications. Embodiments of the cell culture surface coating of the present invention can be tuned by, for example: changing the modulus of the naturally-occurring or synthetic material, changing the freeze-thaw conditions used to provide the matrix, changing the porosity of the material, crosslinking the polysaccharide compounds, mixing multiple gum compounds together, adding additional compounds to the gum material in the coating material, changing the number or nature of the sidechains present on the backbone of polysaccharide gum material in the coating, substituting sidechains, treating with enzymes to change the chemical nature of the material, or by any other method. Embodiments of the present invention include tunable cell culture coatings. Embodiments of coating materials of the present invention can be “tuned” or are “tunable.”

In embodiments of the present invention, the number and nature of galactose sidechains in a polysaccharide cell culture surface coating may be changed. This modification affects the properties of galactose-presenting polysaccharide-based matrices. For example, in embodiments of the present invention, by adding and removing galactose polysaccharide sidechains from galactomannan polysaccharide gum cell culture surface coatings, and by changing the number of mannose groups per galactose in a galactomannose polysaccharide, and/or by changing the nature of these sidechains, polysaccharide cell culture surface coatings including galactomannan polysaccharide coatings are tunable.

In another embodiment, the cell culture surface coating has been treated or tuned to make a physically crosslinked system. A crosslinked gum is a physical gel which can be a gel, a hydrogel, a film or a hydrofilm. A hydrofilm is a thin, transparent or nearly transparent coating. Physical and chemical treatments may cause a cell culture surface coating to become cross-linked. Physical treatments include exposure to particular temperature ranges, including heat, cold and freeze-thaw heat and cold cycles. Solutions of locust bean gum will gel under cryogenic treatment (freezing-thawing cycles). These freeze-thaw cycles create physical-gels or physically crosslinked networks. A solution of locust bean gum, if kept at room temperature will remain fluid. After prolonged storage (2-3 months) the solution will form a weak physical gel. Embodiments of the present invention include gum solutions which have been treated or tuned to form a physical gel.

Embodiments of the present invention include cell culture surface coatings which are tuned to vary the modulus of the polymer coating, using chemical cross-linking chemistry which may change the modulus of the cell culture surface. Cross-linking methods include UV-induced cross-linking, and chemical cross-linking. Chemical agents such as borax (sodium borohydrate), gluteraldedye, epoxy derivatives, and other methods known in the art can be used. UV crosslinking methods also can be employed where a photoinitiator can be used in the gum, photoinitiators attached chemically to the gum or in the blend of gums to initiate gelling or cross-linking behavior. Embodiments of tuned gums of the present invention are gums which have been centrifuged, filtered, heated, frozen, freeze-thawed, chemically or enzymatically treated, exposed to light or otherwise altered, to improve the cell culture characteristics of a cell culture substrate coating made from the treated gum. Tuned gums of the present invention also include gums which are in gel, hydrogel, film or hydrofilm form. Further, tuned gums of the present invention include galactomannan gums which have been centrifuged, filtered, heated, frozen, freeze-thawed, chemically or enzymatically treated, exposed to light or otherwise altered, and which are in gel, hydrogel, film or hydrofilm form for use as a coating for cell culture. In addition, embodiments of the present invention include coatings which are not smooth, but which are “bumps” or “pellets” of gum material on a cell culture surface.

Embodiments of the present invention include mixtures of gums. Mixtures of gums can form cross-linked gel compositions. For example, charged polysaccharides such as xanthan gum or carrageenan or neutral polysaccharides with gelling behavior, such as agar or curdlan, can be mixed with galactomannan gums to form a blend that forms a gel at room temperature.

Embodiments of the present invention also include cell culture surface coatings which have been tuned to change the charge characteristics of the coating, which may be accomplished by changing the charge blending linear or branched charged polysaccharides in the coating. Charged polysaccharides such as xanthan gum or carrageenan are mixed with a naturally occurring gum(s) to form a blend. Combinations of galactomannan gums with other gums such as carageenan and xanthan gums (charged polysaccharides) are capable of synergistic interactions. These gums in combination with locust bean gum form thermoreversible soft elastic gels without any cryogenic (freeze-thaw) treatment. A greater proportion of guar gum (80:20) is required for optimal synergy for room temperature gellation compared to locust bean gum (50:50). In embodiments, the present invention provides mixtures of guar gum; charged polysaccharide ranging from 70:30 to 90:10, and mixtures of locust bean gum; charged polysaccharides ranging from 40:60 to 60:40.

In another embodiment, a mixture (or blend) of at least two types of gum polysaccharides can be used to coat the surface of a substrate to form a cell-culture-friendly coating. Coating a substrate with these gum polysaccharides individually or in a mixture of any combination of these polysaccharides can result in a matrix presenting varied amounts of galactose or other polysaccharide moieties. Mixtures or blends of at least two gums may include, for example, mixtures of any two or more gums from the following: guar gum, locust bean gum (also known as carob bean bum, carob seed gum and carob gum), cassia gum, tragacanth gum, tara gum, karaya gum, gum acacia (also known as gum arabic), ghatti gum, cherry gum, apricot gum, tamarind gum, mesquite gum, larch gum, psyllium, fenugreek gum and tara gum. Gums that are derived from bacterial and algal sources include xanthan gum, seaweed gum, gellan gum, agar gum, carrageenan and curdlan.

In an alternative embodiment, the present invention provides a method to further tune the physical properties of a polysaccharide gum matrix. In one embodiment, conventional cross-linking methods are used to produce the polysaccharide matrix with varying modulus and surface energy (i.e., hydrophilicity). The cross-linking methods include physical, UV-induced cross-linking, and chemical cross-linking. In another embodiment, charged polysaccharides such as xanthan gum or carrageenan are mixed with a naturally occurring gum(s) to form a blend. The resultant blend is used to form a matrix having controlled charge density for cell culturing, including hepatocyte culturing. In another embodiment, linear neutral polysaccharides with gelling behavior, such as agar or curdlan, are mixed with the naturally occurring branched polysaccharide(s) to form a blend. The resultant blend is used to form a matrix having material properties such as modulus and stability for long term cell culturing.

Additional embodiments of the present invention include materials that are gel, hydrogel, film or hydrofilm. The material may be opaque to transparent. Unprocessed and/or higher concentration gum material which contains impurities may form an opaque coating on a cell culture surface, while a more processed and/or lower concentration (i.e. centrifuged, filtered, or chemically purified) material may form a more transparent coating. The concentration of the gum material in solution before the application of the solution to a cell culture vessel may determine whether the material is a gel, hydrogel, film or hydrofilm. A hydrogel or a hydrofilm may be a gel or film which has more water in the structure.

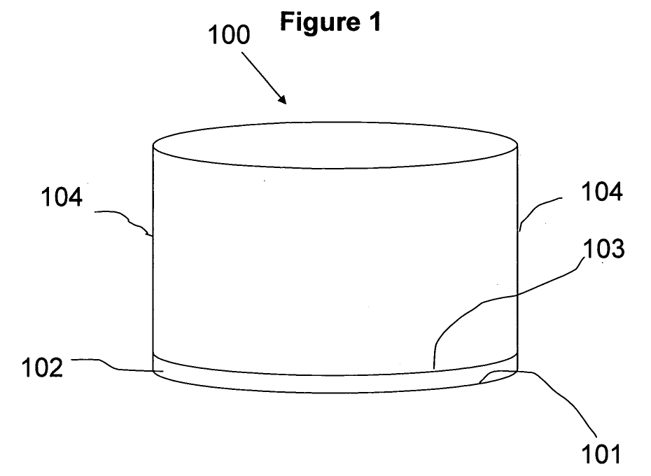

In embodiments, the cell culture coating of the present invention is a thin coating. That is, the coating may be less than 2 mm, less than 1 mm, less than 0.5 mm, less than 0.25 mm, less than 0.1 mm, or less than 0.05 mm in thickness. For example, coatings made from 7.5 mg/ml solutions of locust bean gum (0.75% solution) were measured at approximately 40 μm thick when wet, and approximately 20 μm thick when dry. These measurements may be average measurements taken from multiple sites. The cell culture coating of the present invention is a thin coating, as distinguished from a thick layer of material. Coatings thicker than the disclosed ranges thickness may not stick to the substrate. That is, thicker coatings may delaminate from the substrate and float when aqueous media is introduced into a cell culture vessel containing an embodiment of a cell culture coating of the present invention. In addition, in embodiments of the present invention, the cell culture coating is a continuous coating across the growth surface of a cell culture vessel. That is, the cell culture coating may extend from wall to wall of a cell culture vessel. Embodiments of the cell culture coating of the present invention may not have recesses or hollows. FIG. 1 illustrates a cell culture vessel 100 which has a bottom surface 101, walls 104, and an embodiment of a cell culture surface coating 102 of the present invention. The cell culture surface coating 102 of the present invention has a cell culture surface 103 upon which cells can settle and grow. In embodiments, this cell culture coating 102 provides surfaces 103 which are suitable for growing adherent cells such as hepatocytes. While these cell culture coatings 102 may have some surface topology, in embodiments, these surfaces may be relatively flat.

Embodiments of the present invention include plastic or polymeric substrates which form cell culture substrates or surfaces or vessels or containers and include those comprising or composed of polyester, polystyrene, polypropylene, polymethyl methacrylate, polyolefin, cyclic polyolefin, polyvinyl chloride, polymethyl pentene, polyethylene, polycarbonate, polysulfone, polystyrene copolymers (e.g., SAN and ABS), polypropylene copolymers, ethylene/vinyl acetate copolymer, polyamides, fluoropolymers, polyvinylidene fluoride, polytetrafluoroethylene, silicones, and elastomers, including silicone, hydrocarbon, fluorocarbon elastomers or any other suitable surface. Additional embodiments include plastic or polymeric substrates which have been treated with, for example, plasma.

Embodiments of the present invention include inorganic substrates which form cell culture surfaces or vessels or containers and include those comprising or composed of inorganic materials such as metals, semiconductor materials, glass and ceramic materials. Examples of metals that can be used as surface or substrate materials include oxides of gold, platinum, nickel, palladium, aluminum, chromium, steel, and gallium aresenide. Semiconductor materials used for substrate or surface material can include silicon and germanium. Glass and ceramic materials that can be used for surface or substrate material can include quartz, glass, porcelain, alkaline earth aluminoborosilicate glass and other mixed oxides. Further examples of inorganic substrate materials include graphite, zinc selenide, mica, silica, lithium niobate, and inorganic single crystal materials.

Embodiments of the present invention include labware or cell culture vessels made from a substrate or combination of substrates, where any part of the labware or cell culture vessel has been coated with a cell culture coating of the present invention. Labware includes slides, cell culture plates, multi-well plates, 6-well plates, 12-well plates, 24-well plates, 48-well plates, 96-well plates, 384-well plates, 136 well plates, flasks, multi-layer flasks, bioreactors, cell culture inserts such as the Transwell® made by Corning Incorporated, and other surfaces or containers or vessels useful for cell culture.

Alternate embodiments of the present invention include gum or naturally occurring gum or galactomannan gum-coated cell culture surfaces for use in culturing cells including, but not limited to, stem cells, committed stem cells, differentiated cells, and tumor cells. Examples of stem cells include, but are not limited to, embryonic stem cells, bone marrow stem cells and umbilical cord stem cells. Other examples of cells used in various embodiments include, but are not limited to, myoblasts, neuroblasts, fibroblasts, glioblasts, germ cells, hepatocytes, chondrocytes, keratinocytes, smooth muscle cells, cardiac muscle cells, connective tissue cells, glial cells, epithelial cells, endothelial cells, hormone-secreting cells, cells of the immune system, and neurons. In one aspect, bone cells such as osteoclasts, osteocytes, and osteoblasts can be cultured with the coated substrates produced herein.

Cells useful herein can be cultured in vitro, derived from a natural source, genetically engineered, or produced by any other means. Any source of cells can be used. Atypical or abnormal cells such as tumor cells can also be used herein. Tumor cells cultured on coated substrates described herein may provide more accurate representations of the native tumor environment in the body for the assessment of drug treatments. Growth of tumor cells on the substrates described herein may facilitate characterization of biochemical pathways and activities of the tumor, including gene expression, receptor expression, and polypeptide production, in an in vivo-like environment allowing for the development of drugs that specifically target the tumor.

Cells that have been genetically engineered can also be used. Engineering involves programming the cell to express one or more genes, repressing the expression of one or more genes, or both. Genetic engineering can involve, for example, adding or removing genetic material to or from a cell, altering existing genetic material, or both. Embodiments in which cells are transfected or otherwise engineered to express a gene can use transiently or permanently transfected genes, or both. Gene sequences may be full or partial length, cloned or naturally occurring.

Hepatocytes have been shown here as an exemplary cell type. Although hepatocytes are shown, any other cell or cell type may be grown in culture using embodiments of the present invention. Hepatocytes represent significant cell culture challenges because long term cell culture of primary and secondary hepatocytes generally results in cells which lose their physiological characteristics over time. Primary hepatocytes are anchorage dependent cells and are very difficult to maintain in vitro, losing their adult liver morphology and differentiated functions when cultured in monolayers or suspension. Several cell culture models are being investigated based on complex hepatocyte microenvironment. These include studying various components in extracellular matrix, modifying cell culture media with addition of low concentrations of hormones, corticosteroids, cytokines, vitamins, or amino acids, and understanding cell-cell interactions, both homotypic (hepatocyte-hepatocyte) and heterotypic (i.e. hepatocyte-nonparenchymal cell). All these have shown that hepatocyte function and proliferation in vitro are modulated by cell attachment, cell shape and cell spreading through cell-cell and cell-matrix interactions and that the hepatocyte behavior in vitro can be modified by the culture medium and the cell culture substrate. It is desirable to develop cell culture conditions that provide difficult to culture cells like hepatocytes with an environment that allows the cells to maintain in vivo-specific functions (in the case of hepatocytes, liver-specific functions) in primary and secondary culture achieved by using non-animal derived materials and suitable culture media. For example, on untreated normal polystyrene 2D surface such as tissue culture treated (TCT) polystyrene, hepatocyte cells show flat morphology, great growth rate and low hepatocyte-specific function. By comparison, on Matrigel™ (Becton Dickinson Biosciences), cultured hepatocyte cells show spheroid structure, moderate/low growth rate and high hepatocyte-specific function (data not shown).

For example, an embodiment of the cell culture surface coating of the present invention can be tuned to create conditions that encourage the formation of spheroidal morphology or colony cell culture with hepatocytes or other cell types, and induce the culture of more functionally in-vivo-like cells. These tuned cell culture surface coatings may promote maintenance of cell-cell interactions (e.g. formation of bile canaliculi, cytoskeletal arrangements) and liver specific cell function (e.g. albumin production) in hepatocytes. For example, by using varied galactose substitutions, the stiffness and solubility of the resultant matrix can be controlled or tuned. The higher the substitution, the higher the stiffness, and the better the solubility, dispersiveness and emulsification (i.e., decreased flexibility). Embodiments of the present invention provide a polysaccharide matrix for more in vivo-like hepatocyte cultures with extended viability (in the differentiated state).

Embodiments of the present invention include cell culture surface coatings which include at least one biologically active molecule. Inclusion of a biologically active molecule may promote cell adhesion, proliferation or survival. Or, the inclusion of a biologically active molecule might improve function of cells in culture. Bioactive molecules include human or veterinary therapeutics, nutraceuticals, vitamins, salts, electrolytes, amino acids, peptides, polypeptides, proteins, carbohydrates, lipids, polysaccharides, nucleic acids, nucleotides, polynucelotides, glycoproteins, lipoproteins, glycolipids, glycosaminoglycans, proteoglycans, growth factors, differentiation factors, hormones, neurotransmitters, pheromones, chalones, prostaglandins, immunoglobulins, monokines and other cytokines, humectants, minerals, electrically and magnetically reactive materials, light sensitive materials, anti-oxidants, molecules that may be metabolized as a source of cellular energy, antigens, and any molecules that can cause a cellular or physiological response. Any combination of molecules can be used, as well as agonists or antagonists of these molecules. Glycoaminoglycans include glycoproteins, proteoglycans, and hyaluronan. Polysaccharides include cellulose, starch, alginic acid, chytosan, or hyaluronan. Cytokines include, but are not limited to, cardiotrophin, stromal cell derived factor, macrophage derived chemokine (MDC), melanoma growth stimulatory activity (MGSA), macrophage inflammatory proteins 1 alpha (MIP-1 alpha), 2, 3 alpha, 3 beta, 4 and 5, interleukin (IL) 1, IL-2, IL-3, IL-4, IL-5, IL-6, IL-7, IL-8, IL-9, IL-10, I-11, IL-12, IL-13, TNF-alpha, and TNF-beta. Immunoglobulins useful herein include, but are not limited to, IgG, IgA, IgM, IgD, IgE, and mixtures thereof. Amino acids, peptides, polypeptides, and proteins can include any type of such molecules of any size and complexity as well as combinations of such molecules. Examples include, but are not limited to, structural proteins, enzymes, and peptide hormones. These compounds can be mixed with the gum compounds as they are being prepared, or can be added to the surface of the cell culture coating after it has been applied to a cell culture surface.

The term bioactive molecule also includes fibrous proteins, adhesion proteins, adhesive compounds, deadhesive compounds, and targeting compounds. Fibrous proteins include collagen and elastin. Adhesion/deadhesion compounds include fibronectin, laminin, thrombospondin and tenascin C. Adhesive proteins include actin, fibrin, fibrinogen, fibronectin, vitronectin, laminin, cadherins, selectins, intracellular adhesion molecules 1, 2, and 3, and cell-matrix adhesion receptors including but not limited to integrins such as α₅β₁, α₆β₁, α₇β₁, α₄β₂, α₂β₃, and α₆β₄.

The term bioactive molecule also includes leptin, leukemia inhibitory factor (LIF), RGD peptide, tumor necrosis factor alpha and beta, endostatin, angiostatin, thrombospondin, osteogenic protein-1, bone morphogenic proteins 2 and 7, osteonectin, somatomedin-like peptide, osteocalcin, interferon alpha, interferon alpha A, interferon beta, interferon gamma, interferon 1 alpha, and interleukins 2, 3, 4, 5 6, 7, 8, 9, 10, 11, 12, 13, 15, 16, 17 and 18.

The term “growth factor” as used herein means a bioactive molecule that promotes the proliferation of a cell or tissue. Growth factors useful herein include, but are not limited to, transforming growth factor-alpha. (TGF-alpha), transforming growth factor-beta. (TGF-beta), platelet-derived growth factors including the AA, AB and BB isoforms (PDGF), fibroblast growth factors (FGF), including FGF acidic isoforms 1 and 2, FGF basic form 2, and FGF 4, 8, 9 and 10, nerve growth factors (NGF) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor (HGF), glial neurotrophic growth factor (GDNF), stem cell factor (SCF), keratinocyte growth factor (KGF), transforming growth factors (TGF), including TGFs alpha, beta, beta1, beta2, and beta3, skeletal growth factor, bone matrix derived growth factors, and bone derived growth factors and mixtures thereof. Some growth factors can also promote differentiation of a cell or tissue. TGF, for example, can promote growth and/or differentiation of a cell or tissue. Some preferred growth factors include VEGF, NGFs, PDGF-AA, PDGF-BB, PDGF-AB, FGFb, FGFa, HGF, and BGF.

The term “differentiation factor” as used herein means a bioactive molecule that promotes the differentiation of cells or tissues. The term includes, but is not limited to, neurotrophin, colony stimulating factor (CSF), or transforming growth factor. CSF includes granulocyte-CSF, macrophage-CSF, granulocyte-macrophage-CSF, erythropoietin, and IL-3. Some differentiation factors may also promote the growth of a cell or tissue. TGF and IL-3, for example, can promote differentiation and/or growth of cells.

The term “adhesive compound” as used herein means a bioactive molecule that promotes attachment of a cell or tissue to a fiber surface comprising the adhesive compound. Examples of adhesive compounds include, but are not limited to, fibronectin, vitronectin, and laminin.

The term “deadhesive compound” as used herein means a bioactive molecule that promotes the detachment of a cell or tissue from a fiber comprising the deadhesive compound. Examples of deadhesive compounds include, but are not limited to, thrombospondin and tenascin C.

The term “targeting compound” as used herein means a bioactive molecule that functions as a signaling molecule inducing recruitment and/or attachment of cells or tissues to a fiber comprising the targeting compound. Examples of targeting compounds and their cognate receptors include attachment peptides including RGD peptide derived from fibronectin and integrins, growth factors including EGF and EGF receptor, and hormones including insulin and insulin receptor.

In another aspect, described herein are methods for determining an interaction between a cell or cell line and a factor or drug, comprising (a) depositing cells on a coated substrate described herein; (b) contacting the deposited cells with a factor; and (c) identifying a response produced by the deposited cells upon contact with the factor.

With a known cell line immobilized on the coated substrates, it is possible to screen the activity of several drugs when the drug interacts with the immobilized cells. Depending upon the cells and drugs to be tested, the cell-drug interaction can be detected and measured using a variety of techniques. For example, the cell can metabolize the drug to produce metabolites that can be readily detected. Alternatively, the drug can induce the cells to produce proteins or other biomolecules. The substrates described herein provide an environment for the cells to more closely mimic the in vivo nature of the cells in an ex vivo environment.

The substrates can be used in high throughput applications for analyzing drug/cell interactions. Cells can be grown on the coatings of the present invention within multi-well plates used in high throughput applications. Cells can then be exposed to a drug, media can be removed from these high-throughput cultures, and the removed media can be analyzed for the presence of metabolites, proteins or other biomolecules. Increasing the population of cells per well, or increasing the in vivo-like nature of cells in culture, may serve to increase the value of signals measured by these techniques.

Embodiments of the present invention also include hepatocyte cell cultures which can be used as bio-artificial livers for use in compound toxicity evaluation, compound metabolisms, and protein synthesis. Hepatocytes have the ability to metabolize, detoxify, and inactivate exogenous compounds such as drugs and insecticides, and endogenous compounds such as steroids. The drainage of the intestinal venous blood into the liver requires efficient detoxification of miscellaneous absorbed substances to maintain homeostasis and protect the body against ingested toxins. One of the detoxifying functions of hepatocytes is to modify ammonia into urea for excretion.

In another aspect, described herein is method for growing cells or tissue, comprising (a) depositing a parent set of cells on a coated substrate described herein, and (b) culturing the coated substrate with the deposited cells to promote the growth of the cells. In a further aspect, described herein is a method of using the coated cell culture vessel having the steps of: (a) introducing cells to the cell culture vessel; (b) adding cell culture media; and, (c) incubating the cells. It is contemplated that viable cells can be deposited on the coated substrates produced herein and cultured under conditions that promote tissue growth. Tissue grown (i.e., engineered) from any of the cells described above is contemplated with the coated substrates produced herein. The coated substrates can support many different kinds of precursor cells, and the substrates can guide the development of new tissue. The production of tissues has numerous applications in wound healing. It is contemplated that tissue growth can be performed in vivo or ex vivo.

Like hepatocytes, other cell types in culture take on a spheroidal cell morphology as a preferred and more in-vivo-like cell culture state. Therefore embodiments of the cell culture surface coatings of the present invention may be used for multiple cell types. The invention can be used for cell types that prefer the “spheroidal” morphology shown by hepatocytes as a mode of their own function, and by other cell types.

### WORKING EXAMPLES

The following examples are included to demonstrate embodiments of the invention and are not intended to limit the scope of the invention in any way. It should be appreciated by those of skill in the art that the techniques disclosed in the examples which follow represent techniques used by the inventors to function well in the practice the invention. However, those of skill in the art should, in light of the present disclosure, appreciate that many changes can be made in the specific embodiments which are disclosed and still obtain a like or similar result without departing from the spirit and scope of the invention.

### Example 1

### Coating Preparation

### Example 1a

### Locust Bean Gum

An amount of locust bean gum (LBG) weighed powder (white-beige) (Sigma-Aldrich, Catalog # G0753, Lot#125K0091 or Catalog # 62631 Flka Chemicals, Lot #1301452, also available from Hercules Incorporated, Aqualon Division (Wilmington, Del.)) was dispersed in deionized water to form 0.1-0.5 wt % solutions and 1-2 wt % solutions and the pH was adjusted to 7 (although these surfaces can be made with or without adjusting the pH to 7). The solutions were degassed by sonication for about 5 minutes. The vessel was sealed and the inhomogeneous solution was stirred at RT first and then the temperature was increased to 100-120° C. with continuous stirring at that temperature for 15-60 min. The 0.1-0.5 wt % solutions contained an insoluble fraction at this state and the solution remained less viscous. The 1-2 wt % solutions were more viscous, opaque and contained an insoluble fraction.

Sodium benzoate can be added to the solution to prevent bacterial degradation. The locust bean gum weighed powder can be additionally processed by, for example, centrifugation, chemical purification and/or filtration as in, for example, steps disclosed in Pai, et al, Carbohydrate Polymers 49 (2002) 207-216. Centrifugation can be accomplished by centrifuging the locust bean gum solution at, for example, 4000 rpm for 20 minutes to separate out impurities. The supernatant containing the locust bean gum polymer can then be separated from the pellet, which will mainly contain impurities.

The locust bean gum may also be purified chemically by ethanol extraction, with or without a centrifugation step. The locust bean gum solution can be poured into an excess of ethanol, causing the locust bean gum to precipitate. The precipitate can then be lyophilized (at RT for 24 hours). The resulting powder can be further processed by mechanical means such as pulverizing the precipitate to form a fine powder.

### Example 1b

### Guar Gum

An amount of guar gum weighed powder (white to beige color), available from Sigma-Aldrich and TIC-Gums Inc. was prepared in concentrations ranging from 0.05 wt % to 2 wt % solutions in deionized water and the pH was adjusted to 7. The solution was treated as above.

### Example 1c

### Guar Gum and Xanthan Gum

An amount of guar was prepared as above. Xanthan gum weighed powder (white to beige color) available from Sigma-Aldrich was prepared as a 1-2 wt % solution in deionized water and the pH was not adjusted, Warming of the solution dissolved the Xanthan Gum powder into solution. Guar Gum (GG) solution was mixed with Xanthan Gum (XG) solution in 1:4 and 1:1 ratios. The GG/XG mixture was heated and stirred at 60° C., prior to application on a substrate.

### Example 1d

### Guar Gum-Carageenan Mixtures

Guar Gum and Carageenan were prepared as 1-2 wt % solutions. The solutions were mixed as described above. Guar Gum-Curdlan mixtures were also prepared and mixed as described.

### Example 2

### Preparation of Coated Cell Culture Surfaces

Solutions prepared as above were applied to substrates using a pipette. Solutions were typically added to 24 well TCT plates (0.5-2 wt %, 0.5 ml, 1 ml, 2 ml or 3 ml) and kept at 60° C. for 12 h-24 h until the coating on the plate appeared dry. Substrates were then re-hydrated by adding deionized water and surfaces were rinsed with water 3 times and kept at 60° C. for 12 h. Plates were kept at 4° C. till used and sterilized using a UV lamp (366 nm) for 1 h before culture. The resulting cell culture surface coating was less than 1 mm thin opaque film when dry and greater than 1 mm, thin opaque hydrofilm when wet.

Solutions were prepared as above and applied to substrates using a pipette. The coated substrates were kept at 60° C. for 0.5 h and then at 4° C. or RT overnight. This procedure was used with favorable results for the GG/XG coating mixture.

### Example 3

### Freeze-Thaw Gellation

The viscous solutions (1-2 wt % solutions of LBG, GG, GG/XG) were added to TCT plates (24 well: 1 ml, 2 ml or 3 ml and 6 well: 6 ml) and kept at −15° C. for 24 h. In this procedure, the coated substrates were not kept at 60° C. for 12 h-24 h and rehydrated. Coated substrates were thawed to 4° C. (12 h) and again kept in the freezer for 24 h. This was repeated twice. Plates were kept at 4° C. before use and excess solvent was removed using a pipette. Samples were sterilized using a UV lamp (366 nm) for 1 h before culture. This freeze-thaw process causes the gum solution to form a physical gel. This method creates gels or hydrogels.

### Example 4

### Room Temperature Gellation

Gum solutions prepared as mixtures was stirred at 60° C. and poured into 24 well plates using a pipette. Typical volumes range from 0.5-2 ml. Kept at room temperature to gel for 24 h. This procedure was used with GG(guar gum)/XG(xanthan gum) and CG(cassia gum)/XG(xanthan gum) coating mixture. Matrigel™ coated cell culture substrates were made according to protocols provided by Becton Dickonson. Collagen I coated cell culture substrates were made according to methods known in the art.

### Example 5

### Cell Culture

HepG2/C3A cells were obtained from American Type Culture Collection (ATCC). Frozen cell were thawed and cultured in Eagle's Minimum Essential Medium (EMEM) on Corning CellBind surface and kept at incubator under 37 C with 5% CO₂. Cells were plated onto substrates coated with gum coatings, Matrigel™ (substrates coated according to product specifications, purchased from Becton Dickenson, catalog number 354234), collagen I or TCT plates in EMEM containing 10% FBS and 1% antibiotic and were grown at 37° C. with 5% CO₂.

### Example 6

### Characterization of Cell Growth and Function

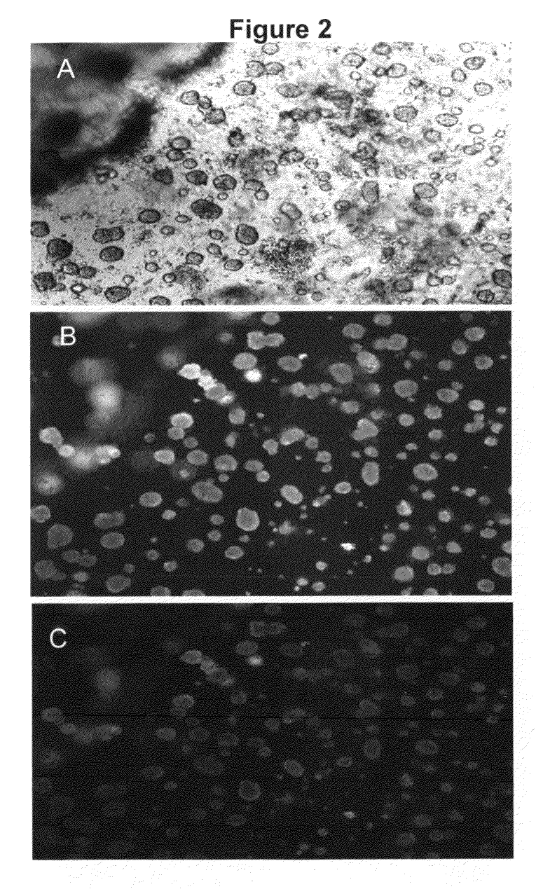

FIG. 2 shows Live/Dead staining of HepG2/C3A cells 9 days after being cultured on 1 wt % locust bean gum-coated surfaces. The cells were stained with a Live/Dead staining reagent kit (live/dead viability/cytotoxicity kit purchased from Invitrogen—catalog number L-3224) and used according to the supplied protocols. Normally, this kit provides fluorescent green (live) and fluorescent red (dead) cell staining. In order to provide black and white figures for the purposes of this disclosure, these figures have been modified to show white areas where either the green or red staining was provided in the original figures. FIG. 2 shows 100-150 μm spheroid structures and almost all of cells are still alive: FIG. 2A is a light microscopy image; FIG. 2B is a fluorescence image at FITC (white spots which were originally stained fluorescent green) channel depicting live cells; FIG. 2C shows fluorescence image at Cy3 (red in original staining) channel depicting dead cells, note that no brightly fluorescent dead cells appear. The white spots visible in FIG. 2B indicate that the live cells are alive and functional. The matrix was formed using 1 w/v % locust bean gum. Both light microscopy and fluorescence images indicate that there are a great numbers of hepatocyte cell spheroid clusters on the matrix. Almost all cells are alive after 14 days culturing (data not shown). FIG. 2 demonstrates the long-term culture of proliferating hepatocytes that retain hepatic function to produce a hepatic cell culture on these locust bean gum matrixes.

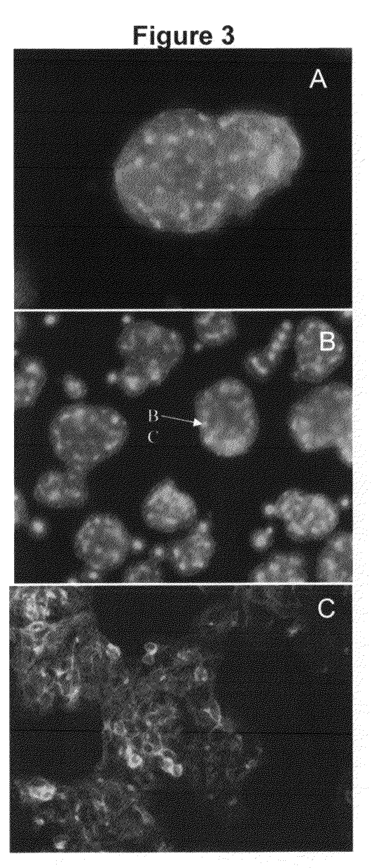

FIGS. 3A, B and C illustrate actin filament staining of HepG2/C3A cells with Texas Red-phalloidin of cells 9 days after being cultured on distinct surfaces. Again, while these images so treated show red staining, for the purposes of this disclosure, the red staining is shown by white areas. FIG. 3A shows locust bean gum-coated substrate (at 40× magnification). FIG. 3B shows Matrigel™ substrate (at 20× magnification). FIG. 3C shows cells growing on a TCT plate (40× magnification). Fluorescence images indicated that the live cells are mostly in spheroids on both the locust bean gum and Matrigel™ surfaces, but not on TCT surface. Bile Canaliculi structure (BC), a morphological characteristic of functional live cells, can be evidenced on the both locust bean gum and Matrigel™ surfaces, but not on TCT surface.

FIGS. 3A, 3B and 3C show that the hepatocyte cells growing on embodiments of coated surfaces of the present invention are in spheroid shape with bile canaliculi structures, indicating that the hepatocytes are capable of secreting proteins and lipids synthesized, and these cells are functional, unlike on the TCT surface where the cells are flat and actin filaments are primarily elongated. The live cells are mostly in spheroids on both the locust bean gum and Matrigel™ surfaces.

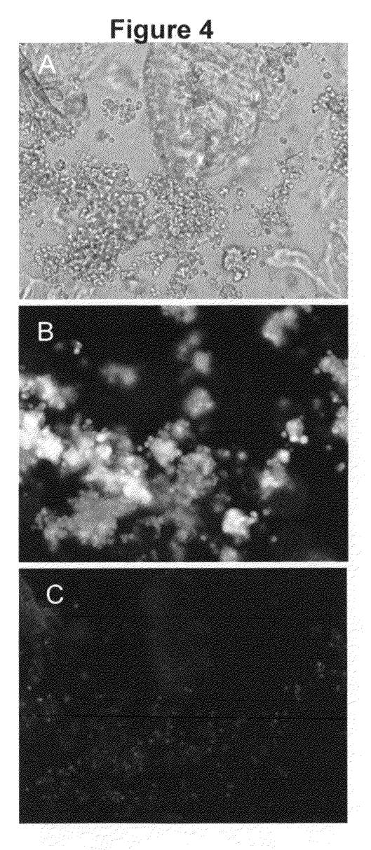

FIGS. 4A, 4B and 4C illustrate microscopic images of HepG2/C3A cells grown on GG/XG (4:1) cell culture coating, unstained and after Live/Dead staining, respectively, as described above. FIG. 4A illustrates that cells grown on guar gum/xanthan gum (GG/XG) (4:1) exhibited different cell morphology from that shown after culturing on locust bean gum coatings, Matrigel™ or TCT, but that the individual cells exhibited rounded aggregated cell structure. Some dead cells were observed, see FIG. 4C.

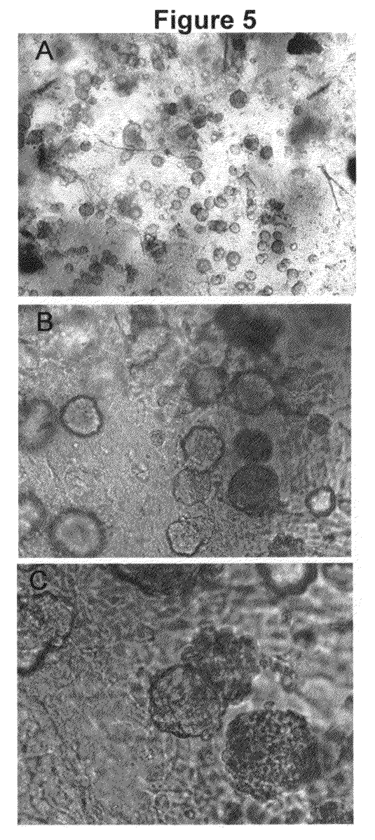

FIGS. 5A, 5B and 5C illustrate bright field photomicrographs, shown at increasing magnifications (5×, 20× and 40×, respectively) illustrating HepG2/C3A cells grown on a Guar Gum/Curdlan cell culture coating. FIG. 5 illustrates that cells grown on this coating appear to be spheroid cells.

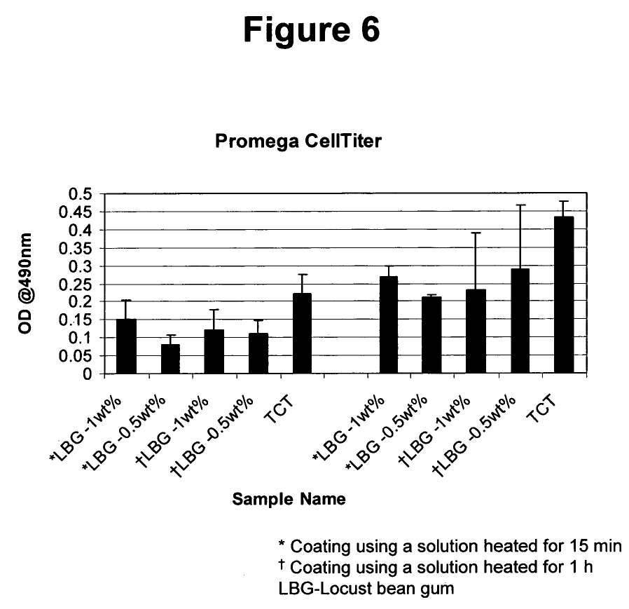

FIG. 6 illustrates cell viability and growth assays of hepatocytes 1 day and 3 days after being cultured on locust bean gum (LBG) -coated cell culture substrates. FIG. 6 shows cell growth following hepatocyte culture on 1 wt % and 0.5 wt % locust bean gum (LBG)-coated TCT substrate and uncoated TCT culture plates. A Promega CellTiter assay kit was used based on the standard protocols provided by Promega. Results, as illustrated in FIG. 6, showed that hepatocyte cells exhibit moderate growth on surfaces coated with locust bean gum (LBG), but not as great as on TCT. (* indicates a coating using a solution heated for 15 minutes, + indicates a coating using a solution heated for 1 hour, LBG is locust bean gum). Matrigel™ exhibits moderate growth, similar to that shown on LBG, and not as great as that shown on TCT (data not shown). Typically hepatocytes that remain round in a spheroidal morphology, exhibit increased differentiated functions and a concomitant decrease in cell proliferation.

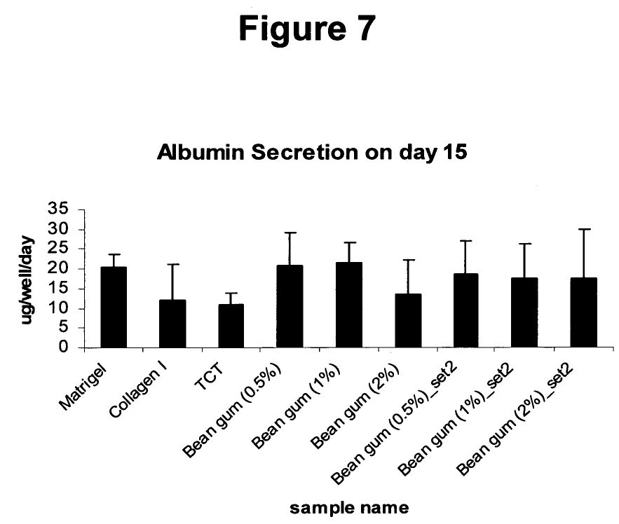

FIG. 7 illustrates the results of albumin assays performed on HepG2/C3A cells grown on locust bean gum-coated cell culture surfaces, compared to Matrigel™ collagen I, and TCT. HepG2/C3A cells were grown for 7 days, 10 days and 14 days. Supernatant/media was harvested on day 7, day 10 and day 14 and examined for the presence of albumin using a Competitive Elisa assay (Biomeda Micro-Albumin Quantitative Test kit, catalog number EU1057) according to manufacturer's protocols. Albumin is a common protein that functional liver cells (HepG2/C3A cells) produce in vivo. FIG. 7 illustrates that cells grown on locust bean gum excreted albumin in an amount similar to that exhibited by cells grown on Matrigel™. TCT-grown cells excreted less albumin than either the Matrigel™ or the locust bean gum-coated surfaces. The total amount of albumin produced increases, as the culturing time increases. The total amount of albumin produced for HepG2/C3A cells cultured on all naturally occurring polysaccharide-coated surfaces are higher than that on TCT.

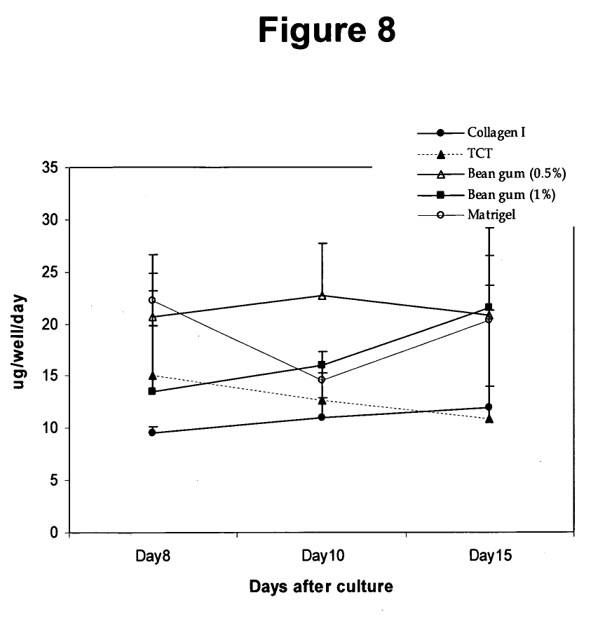

FIG. 8 illustrates albumin production evaluations of HepG2/C3A cells cultured on distinct substrates with Matrigel™, Collagen coated dishes and TCT as controls. The polysaccharide surfaces are locust bean gum-0.5 wt %, and locust bean gum-1 wt % at three different times (8, 10, and 15 days) culturing. Here too the results showed the ability of HepG2/C3A cells to produce albumin on the locust bean gum-coated surfaces. The total amount of albumin produced for HepG2/C3A cells cultured on all bean gum surfaces are higher than that on TCT and collagen coated dishes and remain comparable to Matrigel™. The substrates are not normalized to cell number due to difficulties in quantifying but as shown in FIG. 6, cell growth in TCT is larger than the polysaccharide surfaces.

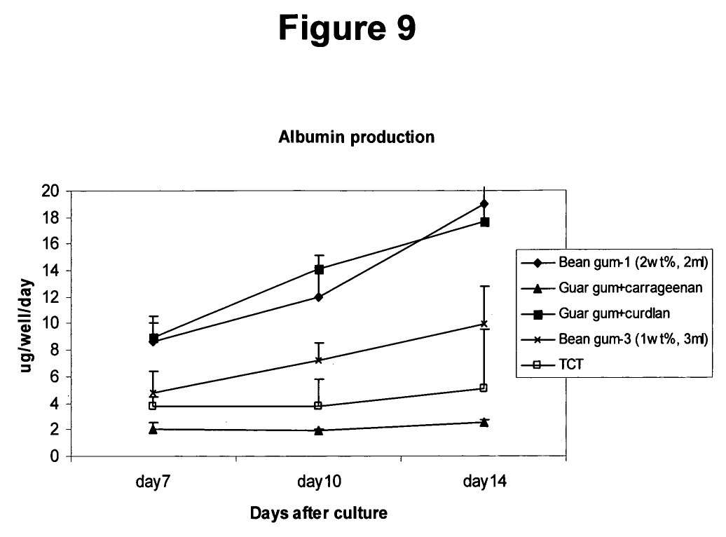

FIG. 9 illustrates albumin production evaluations at three different times (7, 10, and 14 days culturing) for TCT, locust bean gum-1 (2 wt %, 2 ml used), locust bean gum-3 (1 wt %, 3 ml used), a blend of guar gum and carrageenan, and a blend of guar gum and curdlan-coated cell culture surfaces. Results showed that the ability of HepG2/C3A cells to produce albumin is clearly evidenced on all gum or polysaccharide-coated surfaces. The total amount of albumin produced increases, as the culturing time increases. The total amount of albumin produced for HepG2/C3A cells cultured on all gum or polysaccharide-coated surfaces are higher than that on TCT.

The invention and its embodiments being thus described, the same may be varied in many ways by one of ordinary skill in the art having had the benefit of the present disclosure. Such variations are not regarded as a departure from the spirit and scope of the invention, and such modifications are intended to be included within the scope of the following claims and their legal equivalents.

What is claimed is:

1. A cell culture surface coating comprising at least one galactomannan gum wherein the cell culture surface coating is less than 1 mm in thickness.

2. The coating of claim 1 wherein the at least one galactomannan gum is selected from the group consisting of locust bean gum, (carob bean gum, carob seed gum, carob gum), guar gum, cassia gum, tara gum, mesquite gum, and fenugreek gum.

3. The coating of claim 1 wherein the at least one galactomannan gum has a mannose/galactose ratio of from 1:1 to 5:1.

4. The coating of claim 1 wherein the at least one galactomannan gum is locust bean gum.

5. The coating of claim 1 wherein the cell culture surface coating is suitable for growing adherent cells.

6. The coating of claim 1 wherein the cell culture surface coating is suitable for growing hepatocytes.

7. The coating of claim 1 wherein the at least one galactomannan gum is tunable.

8. The coating of claim 1 wherein the at least one galactomannan gum is purified galactomannan gum.

9. The coating of claim 1 wherein the coating is a gel, a hydrogel, a film or a hydrofilm.

10. The coating of claim 1 wherein the coating further comprises a biologically active compound.

11. The coating of claim 10 wherein the biologically active compound comprises amino acids, peptide, polypeptides, proteins, carbohydrates, lipids, polysaccharides, nucleic acids, nucleotides, polynucleotides, glycoproteins, lipoproteins, glycolipids, glycosaminoglycans, proteoglycans, growth factors, differentiation factors, hormones, neurotransmitters, pheromones, chalones, prostaglandins, immunoglobins, monokines, cytokines, humectants, fibrous proteins, or adhesion/deadhesion compounds.

12. A cell culture surface coating comprising at least two gums selected from the group consisting of locust bean gum (carob bean gum, carob seed gum, carob gum), guar gum, cassia gum, tragacanth gum, tara gum, karaya gum, gum acacia (gum Arabic), ghatti gum, cherry gum, apricot gum, tamarind gum, mesquite gum, larch gum, psyllium, fenugreek gum, xanthan gum, seaweed gum, gellan gum, agar gum, cashew gum, carrageenan and curdlan wherein the cell culture surface coating is less than 1 mm in thickness.

13. The coating of claim 12 wherein the coating is a gel, a hydrogel, a film or a hydrofilm.

14. The coating of claim 12 wherein the coating is tunable.

15. The coating of claim 12 wherein the coating further comprises a biologically active compound.

16. The coating of claim 15 wherein the biologically active compound comprises amino acids, peptide, polypeptides, proteins, carbohydrates, lipids, polysaccharides, nucleic acids, nucleotides, polynucleotides, glycoproteins, lipoproteins, glycolipids, glycosaminoglycans, proteoglycans, growth factors, differentiation factors, hormones, nurotransmitters, pheromones, chalones, prostaglandins, immunoglobins, monokines, cytokines, humectants, fibrous proteins, or adhesion/deadhesion compounds.

17. A cell culture vessel for adherent cell culture comprising:
(a) a substrate;
(b) a coating on the substrate wherein the coating comprises at least one galactomannose gum, wherein the coating is less than 1 mm thick.

18. The cell culture vessel of claim 17 wherein the cell culture vessel is for the culture of hepatocytes.

19. The vessel of claim 17 wherein the at least one galactomannan gum is selected from the group consisting of locust bean gum, (carob bean gum, carob seed gum, carob gum), guar gum, cassia gum, tara gum, mesquite gum, and fenugreek gum.

20. The cell culture vessel of claim 17 wherein the galactomannan gum is locust bean gum.

21. The cell culture vessel of claim 17 wherein the substrate comprises plastic or glass.

22. The cell culture vessel of claim 17 wherein the vessel comprises labware.

23. A method of making the cell culture vessel of claim 17 comprising:
(a) preparing a galactomannan gum solution, and
(b) applying a coating of the galactomannan gum solution to the cell culture vessel surface to form a cell culture coating having less than 1 mm in thickness.

24. The method of claim 23, further comprising: after step b, submitting the cell culture vessel to a freeze-thaw cycle.

25. A method of using the cell culture vessel of claim 15 comprising the steps of: (a) introducing hepatocyte cells to the cell culture vessel; (b) adding cell culture media; (c) incubating the cells.
